(19) United States
(12) Patent Application Publication
(10) Pub. No.: US 2004/0052769 A1
(43) Pub. Date: Mar. 18, 2004

(54) Methods of utilizing cultured non-gvhd inducing t lymphocytes to treat disease

(76) Inventor: Yair Reisner, Tel Aviv (IL)

Correspondence Address: GE Ehrlich, Antohny Castorina, 2001 Jefferson Davis Highway, Suite 207, Arlington, VA, 22202, US

(21) Appl. No.: 10/416,916

(22) PCT Filed: Nov. 29, 2001
(86) PCT No.: PCT/IL01/01101

(51) Int. Cl. A61K 45/00; C12N 5/08

(52) U.S. Cl. 424/93.7; 435/372

(57) ABSTRACT

A method of treating a disease in a subject by administration of a non-GVHD inducing population of immune effector cells is disclosed. The method is effected by (a) co-culturing: (i) a first cell population comprising cells specifically immunoreactive to an antigen associated with the disease and cells not immunoreactive to the antigen associated with the disease; and (ii) a second cell population comprising cells being non-syngeneic with the subject and non-syngeneic with the first cell population, the second cell population and the culturing conditions being selected so as to induce proliferation of the cells specifically immunoreactive to an antigen associated with the disease; and (b) administering immune effector cells resultant from step (a) to the subject, thereby treating the disease without inducing GVHD.

### FIELD AND BACKGROUND OF THE INVENTION

The present invention relates to methods of treating viral and malignant diseases using adoptive transfer of immune effectors. More particularly, the present invention relates to methods of treating leukemia using adoptive transfer of allogeneic T lymphocytes without inducing graft-versus-host disease (GVHD).

A great number of devastating human diseases, such as viral and malignant diseases, including leukemias, are associated with expression of abnormal cellular proteins as a result of gene mutation or as a result of viral gene expression, in the case of cancer or viral disease, respectively. Such diseases are also often associated with abnormal overexpression of normal proteins which are either normally expressed at low levels or whose expression is normally restricted to specific developmental stages.

B-cell chronic lymphocytic leukemia (B-CLL): B-cell chronic lymphocytic leukemia, a devastating and frequently fatal disease, is the most common form of leukemia in the Western world. The major impact of this disease is further compounded by the fact that its incidence is on the increase in the rapidly aging population of the Western world (reviewed in: Bannerji R and Byrd J C. Curr Opin Oncol 2000 Jan; 12(1):22).

This disease affects B lymphocytes and causes immunosuppression, failure of the bone marrow, and infiltration of malignant cells into organs. Usually the symptoms and the course of the disease will develop progressively. The incidence is approximately 2 per 100,000 and increases with age, 90% of cases are found in people over 50 years old. Many cases are detected by routine blood tests in people with no symptoms. The cause of B-CLL is unknown and no relationship to radiation, carcinogenic chemicals, or viruses has been determined. The disease is more common in Jewish people of Russian or Eastern European descent and is uncommon in the far east. There is no known way to prevent this disorder and prognosis depends on the stage of the disease. For patients with the earliest stages of the disease, half of patients live more than 12 years whereas for the more advanced stages, half of patients may die within 2 years.

An understanding of the molecular biology of B-CLL has led to the appreciation that several different B-cell diseases are represented under this name. Variability in the bcl-2 family of proteins, p53 mutation, or the presence of various chromosomal abnormalities corresponds to variability of the clinical course of disease and response to therapy. Differential expression of cell surface adhesion molecules by B-CLL cells have also been shown to influence clinical outcome, as have the expression of immune regulatory molecules (e.g., CD80, CD40R, CD27 and CD79b). Recent work studying immunoglobulin-heavy chain gene rearrangement postulates at least two subsets of B-CLL originating from different stages of B-cell development and following different clinical courses. Thus, B-CLL is the final consequence of many different molecular perturbations.

T-cell acute lymphocytic leukemia (T-ALL): Acute lymphocytic leukemia is a progressive, malignant disease characterized by large numbers of immature white blood cells that resemble lymphoblasts and whose cause, in most cases, is unknown. Affected ALL cells can be found in the blood, the bone marrow, the lymph nodes, the spleen, and other organs.

The incidence of this highly debilitating disease is 6 out of 100,000 people and it is responsible for 80% of the acute leukemias of childhood, with the peak incidence occurring between ages 3 and 7. ALL also occurs in adults, where it comprises 20% of all adult leukemias. Acute lymphocytic leukemia remains a difficult disease to treat in adults. Allogeneic bone marrow transplantation can cure a subset of patients with ALL, but GVHD transplant-related mortality and disease relapse remain highly problematic.

In acute leukemia, the malignant cell loses its ability to mature and specialize (differentiate) its function. These cells multiply rapidly and replace the normal cells. Bone marrow failure occurs as malignant cells replace normal bone marrow elements. Affected individuals become susceptible to bleeding and infection because the normal blood cells are reduced in number.

Acute lymphocytic leukemia is treated with a combination of harsh anticancer drugs (chemotherapy) frequently requiring an initial lengthy hospitalization of 3 to 6 weeks for initial chemotherapy. Additionally, patients may require isolation as lymphocyte counts often plummet, putting patients at risk of contracting fatal infections.

Prior Art Methods of Treating Diseases Such as Leukemia Using Adoptive Transfer of Allogeneic T Lymphocytes:

Clinical studies: In one approach, adoptive transfer of peripheral blood mononuclear cells (PBMCs) or HLA-partially matched T cells from in vitro-expanded EBV-specific T-cell lines derived from EBV-seropositive bone marrow (BM) donors has been employed to treat EBV-associated lymphoma in recipients of BM allografts (O'Reilly R J. Important Advances in Oncology 1996:149; O'Reilly R J. Immunological Reviews 1997; 157:195).

In another approach, adoptive transfer of cytotoxic T lymphocytes (CTLs) from HLA-matched, CMV-seropositive donors has been used to treat CMV infection occurring as a result of immunosuppression, similarly to EBV, in post-T cell depleted BMT recipients (Reusser P. Blood. 1991; 78:1373).

Donor-derived CTLs specific for patient-specific minor histocompatibility antigens (mHag's) are involved in mediating GVHD and graft-versus-leukemia (GVL) when treating hematologic malignancies using allogeneic BMT. Minor histocompatibility antigens HA-1 and HA-2 induce HLA-A*0201-restricted CTLs in vivo and are exclusively expressed on hematopoietic cells, including leukemic cells and leukemic precursors, but not on fibroblasts, keratinocytes, or liver cells. Thus, further approaches have utilized targeting mHag's to treat hematological malignancies.

Animal studies: In one animal study employing mHag targeting, CTL clones specific for mHag's were used for inhibiting in vivo engraftment of human acute myeloid leukemia (AML) cells in non-obese diabetic/SCID mice (Bonnet D. Proc Natl Acad Sci USA. 1999; 96:8639).

In vitro studies: Donor-derived CTLs specific for mHag's play an important role in both GVHD and GVL reactivities. mHag's HA-1 and HA-2 induce HLA-A⁰²⁰¹-restricted CTLs in vivo and are exclusively expressed on hematopoietic cells, including leukemic cells and leukemic precursors, but not on fibroblasts, keratinocytes, or liver cells.

Thus, one approach has employed targeting of mHag using HA-1- and HA-2-specific CTLs generated ex vivo from mHag HA-1- and/or HA-2-negative allogeneic donors primed with autologous peptide-pulsed dendritic cells (DCs) for killing primary AML and ALL cells in vitro (Mutis T. Blood 1999; 93:2336).

Another approach has employed targeting of the tumor-overexpressed protein cyclin D1, which is normally expressed at low levels, using anti-cyclin-D1 allogeneic CTL raised by stimulation of PBMCs from allogeneic HLA-A2⁻ donors with the HLA-A2⁺, TAP-deficient human cell line T2 pulsed with cyclin D1 peptides (Sadovnikova E. Eur J. Immunol. 1998; 28:193).

Yet another approach has used targeting of the tumor-overexpressed protein Wilm's tumor-1 (WT1) (Ohminami H. Blood 2000; 95:286; Gao L. Blood 2000; 95:2198), a gene preferentially expressed in acute leukemia cells, regardless of leukemia subtype and chronic myelogenous leukemia (CML) cells in blast crisis, but not in normal cells.

For example, allogeneic anti-WT1 CTL clones generated by stimulating CTL with autologous DCs loaded with a WT1-derived 9-mer peptide consisting of the HLA-A24 (HLA-A*2402)-binding motifs and used to target HLA-A24-positive leukemia cells expressing WT1 (Ohminami H. Blood 2000; 95:286).

All of the above-mentioned approaches, however, suffer from many serious disadvantages.

In the first place, all of the above studies, proposing the use of allogeneic T lymphocytes to kill pathogenic cells, do not provide any satisfactory means to avoid GVHD. The prior art approach to such avoidance of GVHD relies on the use of powerful immunosuppressant drugs. The use of such drugs fails to eliminate potentially immunoreactive clones from the circulation, causes profound immunosuppression, thereby placing patients in grave danger of contracting lethal infections and presents the unfortunate side-effect of causing unacceptable levels of organ damage.

Furthermore, none of the above studies has demonstrated satisfactory in vivo therapeutic effectiveness.

All of the in vitro studies described above suffer from the severe limitations inherent to such studies. Namely, in vitro studies cannot even begin to reproduce the vastly complex physiological, pathological and immunological dynamics found in vivo. As such, approaches thought to be effective in in vitro studies are notoriously often found to be totally ineffective in animal models, not to mention in human therapeutic contexts.

All of the animal models employed by the prior art have failed to demonstrate effective treatment of human disease using long-term functional allogeneic human effectors in the context of a complete immune system and complete avoidance of GVHD.

Finally, approaches employed in human clinical studies have the disadvantage of often being non-randomized, small and employing heterogeneous groups of patients and from being unsatisfactorily ineffective.

Thus, all prior art approaches have failed to provide an adequate solution for treating viral or malignant diseases with allogeneic T lymphocytes in the absence of GVHD.

There is thus a widely recognized need for, and it would be highly advantageous to have, a method of treating diseases using allogeneic or syngeneic lymphocytes which is easily practicable in humans and yet be capable of treating diseases, such as leukemia, without risk of GVHD.

### SUMMARY OF THE INVENTION

According to the present invention there is provided a method of treating a disease in a subject by administration of a non-GVHD inducing population of immune effector, the method comprising: (a) co-culturing: (i) a first cell population comprising cells specifically immunoreactive to an antigen associated with the disease and cells not immunoreactive to the antigen associated with the disease; and (ii) a second cell population comprising cells being non-syngeneic with the subject and non-syngeneic with the first cell population, the second cell population and the culturing conditions being selected so as to induce proliferation of the cells specifically immunoreactive to an antigen associated with the disease; and and (b) administering immune effector cells resultant from step (a) to the subject, thereby treating the disease without inducing GVHD.

According to further features in preferred embodiments of the invention described below, the co-culturing is effected under conditions inducing selective proliferation of the immune effector cells.

According to still further features in the described preferred embodiments, the treating is performed prior to, concomitantly with or following transplantation of allogeneic cells, tissues or organs into the subject.

According to still further features in the described preferred embodiments, the allogeneic cells comprise bone marrow cells.

According to still further features in the described preferred embodiments, the disease is a malignant disease.

According to still further features in the described preferred embodiments, the disease is a viral disease.

According to still further features in the described preferred embodiments, the disease is an autoimmune disease.

According to still further features in the described preferred embodiments, the disease is a leukemia.

According to still further features in the described preferred embodiments, the disease is a myeloid leukemia.

According to still further features in the described preferred embodiments, the disease is a lymphocytic leukemia.

According to still further features in the described preferred embodiments, the disease is an acute leukemia.

According to still further features in the described preferred embodiments, the disease is a chronic leukemia.

According to still further features in the described preferred embodiments, the disease is a T cell leukemia.

According to still further features in the described preferred embodiments, the disease is a B cell leukemia.

According to still further features in the described preferred embodiments, the disease is a T-ALL.

According to still further features in the described preferred embodiments, the disease is a B-CLL.

According to still further features in the described preferred embodiments, the subject is human.

According to still further features in the described preferred embodiments, the administration is effected via intraperitoneal injection.

According to still further features in the described preferred embodiments, the administration is effected via intravenous injection.

According to still further features in the described preferred embodiments, the first cell population is derived from a donor being allogeneic with the subject.

According to still further features in the described preferred embodiments, the first cell population is derived from a donor being syngeneic with the subject.

According to still further features in the described preferred embodiments, the first cell population is derived from the subject.

According to still further features in the described preferred embodiments, the first cell population comprises PBMCs.

According to still further features in the described preferred embodiments, the first cell population comprises PBLs.

According to still further features in the described preferred embodiments, the first cell population comprises cells derived from a lymphoid organ.

According to still further features in the described preferred embodiments, the lymphoid organ is selected from the group consisting of bone marrow, spleen, lymph node, Peyer's patch and thymus.

According to still further features in the described preferred embodiments, the first cell population comprises a population of cells differentiated in vitro.

According to still further features in the described preferred embodiments, the first cell population comprises a population of genetically transformed cells.

According to still further features in the described preferred embodiments, the cells specifically immunoreactive to an antigen associated with the disease comprise T lymphocytes.

According to still further features in the described preferred embodiments, the T lymphocytes comprise helper T lymphocytes.

According to still further features in the described preferred embodiments, the T lymphocytes comprise CTLs.

According to still further features in the described preferred embodiments, the cells not immunoreactive to the antigen associated with the disease comprise T lymphocytes.

According to still further features in the described preferred embodiments, the cells specifically immunoreactive to an antigen associated with the disease are specifically immunoreactive to an antigen associated with the disease as a result of genetic transformation.

According to still further features in the described preferred embodiments, the cells specifically immunoreactive to an antigen associated with the disease comprise genetically transformed natural killer cells.

According to still further features in the described preferred embodiments, the cells not immunoreactive to the antigen associated with the disease are T lymphocytes.

According to still further features in the described preferred embodiments, the second cell population comprises cells being infected with a virus.

According to still further features in the described preferred embodiments, the virus is Epstein-Barr virus.

According to still further features in the described preferred embodiments, the second cell population comprises antigen presenting cells.

According to still further features in the described preferred embodiments, the second cell population comprises B cells.

According to still further features in the described preferred embodiments, the conditions selective for killing cells of the first cell population not immunoreactive to the antigen associated with the disease comprise IL-2 starvation.

According to still further features in the described preferred embodiments, the conditions suitable for selective proliferation of the immune effector cells comprise IL-2 supplementation.

According to still further features in the described preferred embodiments, the conditions suitable for selective proliferation of the immune effector cells comprise co-culture of the immune effector cells with the second cell population.

The present invention successfully addresses the shortcomings of the presently known configurations by providing a method of treating a disease in a subject by administration of a non-GVHD inducing population of immune effector cells.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic diagram depicting the protocol for generation of human/mouse radiation chimeras. Mice from strains with normal immune systems are subjected to lethal split-dose total body irradiation (TBI) on Days −4 and −1. On Day 0, mice are radioprotected with an NOD-SCID BM cell infusion and are transplanted on Days 1 or 7 with fresh human PBMC via the intraperitoneal route, as previously described (Lubin et al. 1994, Blood 83(8):2364-2381).

FIG. 2 is a schematic diagram depicting the protocol for generation of human allogeneic non-alloreactive anti-third party CTLs.

FIGS. 3a-i are FACS analysis data plots depicting engraftment of primary peripheral blood lymphocytes (PBLs) from B-CLL patients in human mouse radiation chimeras at different stages of the disease. Stage 0: FIGS. 3a-c, Stages I-II: FIGS. 3d-f and Stages III-IV: FIGS. 3g-i. Human leucocytes, B-CLL cells and T cells are characterized by CD45+, CD20+CD5+ and CD3+CD5+ phenotypes, respectively. The vertical axes of data plots depict staining for the markers CD45 (FIGS. 3a, 3d and 3g), CD3 (FIGS. 3b, 3e and 3g) and CD20. (FIGS. 3c, 3f and 3i). The horizontal axes of all data plots depict staining for the marker CD5.

FIG. 4 is a histogram depicting engraftment of T-ALL cells in human mouse radiation chimeras during Days 1-9 post transplant. Data depicts the numbers of live and dead cells retrieved from the peritoneum daily.

FIG. 5 is a histogram depicting in vivo eradication of B-CLL cells in human/mouse radiation chimeras by human allogeneic anti-third party CTLs. Results from a representative experiment using B-CLL cells from three different patients are shown.

FIG. 6 is a histogram depicting in vivo eradication of T-ALL cells in human/mouse radiation chimeras by human allogeneic anti-third party CTLs. Results from a representative experiment using B-CLL cells from 3 different patients are shown.

FIG. 7 is a histogram depicting efficient in vitro killing of B-CLL cells (“GVL”) without killing of B-CLL-autologous T cells (“GVHD”) by human allogeneic and B-CLL-autologous anti-third party CTLs.

FIG. 8 is a histogram depicting efficient in vitro killing of T-ALL cells by human allogeneic anti-third party CTLs. T-ALL cells were incubated alone [TALL] or with human allogeneic CTLs [CTL+T-ALL]. A representative experiment is shown.

FIG. 9 is a histogram depicting efficient in vivo killing of leukemia cells by leukemia-autologous human anti-third party CTLs. A representative experiment is shown.

FIG. 10 is a histogram depicting the requirement of cell-cell contact for killing of B-CLL cells by human allogeneic anti-third party CTLs in a transwell assay. A representative experiment is shown.

FIG. 11 is a histogram depicting the requirement of cell-cell contact for killing of B-CLL cells by B-CLL-autologous anti-third party CTLs in a transwell assay. A representative experiment is shown.

FIG. 12 is a histogram depicting extremely killing of T-ALL cells by human allogeneic anti-third party CTLs via soluble factors only in a transwell assay.

### DESCRIPTION OF THE PREFERRED EMBODIMENTS

The present invention is of methods of treating a disease in a subject by administration of a non-GVHD inducing population of cultured immune effector cells. Specifically, the present invention uses cultured allogeneic or syngeneic T lymphocytes possessing enhanced immunoreactivity towards leukemic cells and possessing reduced immunoreactivity towards non-leukemic cells. As such, when administered to a subject, such cultured immune effector cells facilitate treatment of leukemia without inducing GVHD in the subject.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

Various methods of utilizing allogeneic T lymphocytes capable of treating viral or malignant disease have been described by the prior art.

For example, one clinical approach has employed adoptive transfer of allogeneic T cells from EBV- or CMV-seropositive donors to treat EBV-associated lymphoma or CMV infection, respectively, in recipients of BM allografts.

Another approach using an in vivo animal model has employed CTL clones specific for mHag's, a tumor-specific antigen, to inhibit in vivo engraftment of human AML cells in immunocompromised mice.

Numerous in vitro approaches have also utilized targeting of tumor-specific antigens.

For example, one approach has employed targeting of mHag expressing cells using CTLs specific for the mHag's HA-1 and HA-2 being derived from HA-1- and/or HA-2-negative allogeneic donors and being primed with autologous peptide-pulsed DCs so as to target AML and ALL cells.

Another approach has employed anti-cyclin D1 CTLs raised by stimulation of PBMCs with the TAP-deficient human cell line T2 pulsed with cyclin D1 peptides.

Yet another approach has used anti-WT1 CTL clones generated by stimulating CTL with autologous DCs loaded with a WT1 peptide.

However, such prior art approaches do not provide satisfactory means to avoid GVHD when treating disease. Namely, such prior art approaches rely on the use of powerful immunosuppressant drugs which fail to eliminate potentially immunoreactive clones from the circulation, cause profound immunosuppression, thus risking lethal infection, and are associated with unacceptable levels of organ toxicity.

Furthermore, prior art approaches have not demonstrated satisfactory in vivo therapeutic effectiveness and in vitro approaches cannot satisfactorily model the complex in-vivo physiological, pathological and immunological dynamics existing in vivo.

Also, prior art animal models have yet to demonstrate effective treatment of human disease using functional allogeneic human effectors in the context of a complete immune system in the absence of GVHD.

Finally, human clinical studies present the significant drawbacks of being non-randomized, small and of employing heterogeneous groups of patients.

Thus, prior art approaches employing allogeneic T lymphocytes have failed to provide adequate solutions for treating malignant or viral diseases without risk of GVHD.

While reducing the present invention to practice, purified preparations of allogeneic or syngeneic cultured T lymphocytes immunoreactive to antigens associated with a disease were generated under conditions non-permissive to survival of T lymphocytes non-immunoreactive to antigens associated with the disease. Hence, the present invention enables the generation of allogeneic or syngeneic T lymphocyte populations which are highly effective for treating a disease in a subject while being devoid of GVHD inducing cells.

Thus, according to one aspect of the present invention, there is provided a method of treating a disease in a subject by administering to the subject a non-GVHD inducing population of allogeneic or syngeneic immune effector cells.

As is further exemplified in the Examples section which follows, these immune effector cells are generated by co-culturing of a first cell population which includes cells specifically immunoreactive to an antigen associated with the disease (preferably T-lymphocytes) and cells not immunoreactive to the antigen associated with the disease with a second cell population which includes cells that are non-syngeneic with the subject and the first cell population (preferably of an MHC haplotype different from that of the cells of the first population by at least one, preferably at least two, more preferably at least three, more preferably at least four, more preferably at least five, more preferably at least six MHC determinants). In order to facilitate generation of the immune effector cells, the second cell population and the culturing conditions are selected capable of inducing proliferation of the cells specifically immunoreactive to an antigen associated with the disease. The second cell population and the culturing conditions are also preferably selected capable of killing cells of the first cell population not immunoreactive to the antigen associated with the disease. As is illustrated in the Examples section which follows, the second cell population preferably includes EBV infected B-lymphocytes which have been shown herein to be highly effective in performing the above described functions.

To further enhance and/or specifically tailor the therapeutic efficacy of the immune effector cells generated according to the teachings of the present invention, the method of generating such immune effector cells described above can also be effected under conditions suitable for the proliferation of the cells specifically immunoreactive to an antigen associated with the disease. Such conditions are described in detail herein under and in the Examples section which follows.

Since immune effector cells, such as T lymphocytes, have the capacity to be immunoreactive, via T cell receptor (TCR) activation, in response to cells displaying or expressing specific cell-surface molecules, the method according to the present invention can be used to treat diseases associated with cells displaying or expressing specific cell-surface molecules associated with such diseases.

It will be understood by one of ordinary skill in the art that the T lymphocytes of the present invention have the capacity to be immunoreactive to cells expressing or displaying cell-surface molecules associated with a disease such as, for example, polypeptides displayed at the cell surface in a complex, for example, with an MHC molecule. Furthermore, it will also be understood by one of ordinary skill in the art that the T lymphocytes of the present invention have the capacity to be immunoreactive to cells expressing or displaying cell-surface molecules associated with a disease, such as, for example, lipids or glycolipids expressed or displayed at the cell surface in a complex, for example, with CD1 molecules.

Examples of polypeptides associated with a disease include, but are not limited to, non-self polypeptides, mutated self-polypeptides, abnormally expressed or displayed self-polypeptides and self-antigen specific immune recognition molecules.

Examples of non-self polypeptides include, but are not limited to, viral, bacterial, mycoplasma, protozoan or parasitic polypeptides.

Examples of viral polypeptides include, but are not limited to, HIV, CMV, influenza, EBV and rhinovirus polypeptides. According to a preferred embodiment, the method of the present invention is used treat EBV-infected cells.

Examples of HLA-restricted EBV antigens include, for example, antigens derived from the viral proteins EBNA-2 to 6, LMP-1 and LMP-2.

Examples of mutated self-polypeptides include mutated polypeptides associated with malignant diseases, such as, but not limited to, polypeptides derived from p53.

Examples of abnormally expressed or displayed self-polypeptides, include, for example, polypeptides displayed or expressed during an inappropriate developmental stage, such as, for example, carcinoembryonic antigen (CEA) which is expressed in numerous types of cancer, such as, but not limited to gastrointestinal cancer.

Further examples of abnormally expressed or displayed self-polypeptides, include, for example, polypeptides displayed or expressed with inappropriate tissue-specificity.

Yet further examples of abnormally expressed or displayed self-polypeptides, include, for example, polypeptides displayed or expressed at abnormally high levels, such as, for example, HER-2, which is highly overexpressed in many different types of adenocarcinomas.

Examples of lipids or glycolipids associated with a disease include lipids or glycolipids of bacterial or mycoplasma origin.

Examples of self-antigen specific recognition molecules include TCRs, B cell receptors (BCRs) or antibodies involved in mediating autoimmune diseases.

Although the method of the present invention can be generally applied to treat diseases, such as infectious, malignant or autoimmune diseases, which are characterized by cells expressing or displaying specific cell-surface molecules in a complex with specialized antigen-presenting molecules, such as MHC or CD1, the method of the present invention can further be applied to treat diseases characterized by cells expressing or displaying specific cell-surface molecules which are not in a complex with specialized antigen-presenting molecules.

Treatment of diseases characterized by cells expressing or displaying specific cell-surface molecules which are not in a complex with specialized antigen-presenting molecules (e.g. MHC or CD1 etc.) can be effected, for example, in cases where the cells specifically immunoreactive to an antigen associated with a disease of the present invention include, for example, immune effector cells, such as T lymphocytes or NK cells, having been genetically transformed to express or display at the cell surface a receptor possessing direct, non-antigen presenting molecule-restricted, antigen-recognition specificity and a concomitant capacity to activate such an immune effector cell in response to receptor ligation. Examples of such receptors and methods of using such are well known to one skilled in the art and have been extensively described in the literature (Eshhar Z. Cancer Immunol Immunother. 1997 November-December; 45(3-4):131 and Bitton N. et al., Curr Top Microbiol Immunol. 2001;260:271, all of which incorporated herein by reference in their entirety).

Diseases characterized by cells expressing or displaying specific cell-surface molecules which are not in a complex with specialized antigen-presenting molecules, such as MHC or CD1 include, for example, diseases associated with cells in which there is overexpression or excessive cell surface display of self-molecules, overexpression or excessive cell surface display of self-molecules during an inappropriate developmental stage or overexpression or excessive cell surface display of self-molecules with inappropriate cellular or tissue type specificity.

Preferably, the method of the present invention is applied to treat diseases, such as malignant or viral diseases, more preferably diseases being both viral and malignant, such as leukemia associated with viral infection.

Most preferably, the method of the present invention is applied to treat leukemias, such as T-cell acute lymphocytic leukemia (T-ALL) and B-cell chronic lymphocytic leukemia (B-CLL).

As shown in Example 1 of the Examples section which follows, the method of the present invention enables highly effective in vivo treatment of human B-CLL or T-ALL by either leukemia-allogeneic or leukemia-autologous human effectors in the absence of GVHD.

As such, the method of the present invention represents a very marked improvement over prior art methods of treating diseases using autologous or allogeneic T lymphocytes since such methods have not been shown to be satisfactorily effective and/or to satisfactorily reduce the risk of GVHD without relying on highly hazardous immunosuppressive regimens, as described above.

It has been shown by the present inventor in an in vivo model of fully allogeneic BMT that administration of anti-third party CTLs, such as the therapeutic T lymphocytes of the present invention, can induce tolerance to transplants syngeneic with such anti-third party CTLs in sublethally irradiated recipients.

Thus, the method of the present invention can furthermore be advantageously applied towards treating a disease in a subject while concomitantly facilitating engraftment of a transplant of cells, tissues or organs syngeneic with such therapeutic T lymphocytes.

Preferably, this is effected in the context of standard leukemia therapy in which treatment of a subject comprises sub-lethal TBI followed by radioprotection with allogeneic BMT. The therapeutic T lymphocytes of the present invention can thus be administered to such a subject following TBI so as to facilitate eradication of residual leukemic cells while concomitantly facilitating engraftment of a radioprotective BM transplant.

Furthermore, the capacity of the therapeutic T lymphocytes of the present invention to treat leukemia serves to allow treatment with minimal levels of TBI in such a therapeutic modality.

According to the present invention, the method of treating a disease in a subject is effected by co-culturing a cell population comprising immune effector cells under conditions suitable for selectively inducing death of cells which are non-immunoreactive to a set of antigens being allogeneic with the subject and being allogeneic with the cell population comprising immune effector cells, thereby generating a therapeutic immune effector cell population.

As described in detail in the Examples section below, culturing of cell populations comprising immune effector cells to treat a disease according to the method of the present invention generates a population of immune effector cells being enriched for immune effector cells immunoreactive to antigens associated with the disease and being depleted for effector cells immunoreactive to antigens of the subject.

Hence, immune effector cells cultured according to the present invention are highly suitable for treating a disease in a human subject with minimal risk of inflicting GVHD.

According to a preferred embodiment of the present invention, the conditions employed to culture a cell population comprising immune effector cells for treating a disease in a subject include co-culture with a “third-party” cell population being non-syngeneic with the subject and being non-syngeneic with the aforementioned cell population comprising immune effector cells. Culturing of cell populations comprising immune effector cells with such third-party cell populations is described in detail in the Examples section, below.

Since it is well known to those versed in the art that in the absence of activation stimuli, such as via polyclonal activators, such as IL-2, or via antigen-specific TCR signaling, cultured immune effector cells, such as T lymphocytes, do not survive, culturing of immune effector cells to treat a disease in a subject is effected in the absence of such activation stimuli, preferably in the absence of exogenous IL-2 supplementation. This ensures that T lymphocytes non-immunoreactive to third-party cell antigens are depleted, thereby leading to generation of a population of therapeutic immune effector cells enriched with effectors immunoreactive to antigens associated with the disease and depleted of effectors being immunoreactive to antigens of the subject.

According to a preferred embodiment of the present invention, cell populations comprising immune effector cells are co-cultured with third-party cells having been subjected to a growth-arresting treatment, preferably by irradiation with 500-2,000 Gy, more preferably 1,000 Gy.

According to another preferred embodiment of the present invention, third-party cells are cells possessing antigen-presenting functions, such as, for example, dendritic cells, such as peptide-pulsed dendritic cells or B cells.

Preferably the third-party cells of the present invention are B cells.

According to yet another preferred embodiment, the third-party cells of the present invention are infected with a virus, preferably EBV.

According to a most preferred embodiment of the present invention, the third-party cells of the present invention are B cells having been genetically transformed with EBV. Such cells can be used to generate therapeutic immune effector cells suitable for treating a disease, as described in the Examples section which follows.

According to an embodiment of the present invention, co-culturing of cell populations comprising immune effector cells with third-party cells in the absence of exogenous IL-2 supplementation is preferably effected for a period of 7-21 days, more preferably, 10-17 days, most preferably 14 days.

Following co-culturing of a population of cells comprising immune effector cells with third-party cells in the absence of exogenous IL-2 supplementation, the method of the present invention preferably comprises further culturing the resultant therapeutic immune effector cells with exogenous IL-2 supplementation so as to expand the numbers of T lymphocytes immunoreactive to antigens associated with the disease, thereby providing a means of more effectively treating the disease or of treating the disease in cases where the numbers of therapeutic T lymphocytes generated by culturing in the absence of exogenous IL-2 supplementation is insufficient to treat the disease.

Preferably, exogenous IL-2 supplementation is effected by supplementing culture media with 75-1,200 IU/ml IL-2, more preferably 150-600 IU/ml IL-2, most preferably, 300 IU/ml IL-2.

Following generation of therapeutic immune effector cells by culturing in the absence of exogenous IL-2 supplementation, the method of treating a disease of the present invention preferably further comprises culturing such cells in the presence of third-party cells so as to provide activation stimuli, thereby facilitating the aforementioned expansion of therapeutic immune effector cells.

Preferably, following culture in the absence of exogenous IL-2 supplementation, the therapeutic T lymphocytes for treating a disease of the present invention are further cultured with both exogenous IL-2 supplementation and by restimulation with third-party cells, thereby maximizing the aforementioned expansion of therapeutic immune effector cells.

According to a preferred embodiment, the cell population comprising immune effector cells which is cultured, according to the method of the present invention, to generate therapeutic immune effector cells for treating a disease in a subject is derived from a donor being allogeneic with the subject, more preferably from a donor being syngeneic with the subject, such as the subject.

The advantage of using therapeutic immune effector cells being syngeneic with the subject, such as cells from the subject, over using cells being allogeneic with the subject is that cells being syngeneic with the subject minimize the risk of being rejected or of inducing GVHD when administered to the subject. The advantage of using immune effector cells being allogeneic with the subject is advantageous when the number of cells which can be obtained from which to generate therapeutic immune effector cells is limited, such as, for example, when harvesting cells from a B-CLL patient.

According to the present invention, the cell population which is cultured to generate therapeutic immune effector cells can consist of, for example, PBMCs, PBLs, cells derived from a lymphoid organ, BM cells, splenocytes, lymph node cells, Peyer's patch cells, thymocytes or cells differentiated in vitro from precursor cells, such as hematopoietic stem cells or hematopoietic progenitor cells.

Preferably, the cell population which is cultured to generate the therapeutic immune effector cells of the present invention comprises PBMCs, as described in greater detail in the Examples section, below.

Following generation of therapeutic immune effector cells, as described in the Examples section below, the method of treating a disease in a subject is further effected by administering a dose of the therapeutic immune effector cells to the subject.

According to the present invention, administration of therapeutic immune effector cells is preferably effected via the intraperitoneal route, although it will be appreciated to one skilled in the art that administration can be effected through other routes, such as, but not limited to, the intraperitoneal route.

According to a preferred embodiment, the therapeutic immune effector cells of the present invention are T lymphocytes, such as, for example CTLs or helper T lymphocytes.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes 1-111 Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Efficient In Vivo and In Vitro Killing of Human Leukemia Cells by Treatment with Human Allogeneic Anti-Third Party CTLs in the Absence of GVHD

A very large number of diseases, such as viral or malignant diseases should be amenable to treatment using T lymphocytes, as such diseases express distinctive antigens in the context of MHC at the cell surface. The use of allogeneic or syngeneic lymphocytes constitute promising approaches to disease treatment, however, prior art approaches have failed to provide a satisfactory means to prevent GVHD from being induced by administration of allogeneic lymphocytes and, furthermore, prior art approaches have also failed to demonstrate generation of therapeutically effective autologous T lymphocytes.

In order to provide a means of treating viral and malignant disease using either autologous or allogeneic T lymphocytes, the present inventors have demonstrated the capacity of human anti-third party T lymphocytes cultured according to the method of the present invention to eradicate established human B-CLL and T-ALL in the absence of GVHD in a realistic in vivo model of such diseases, as described below.

Methods and Materials:

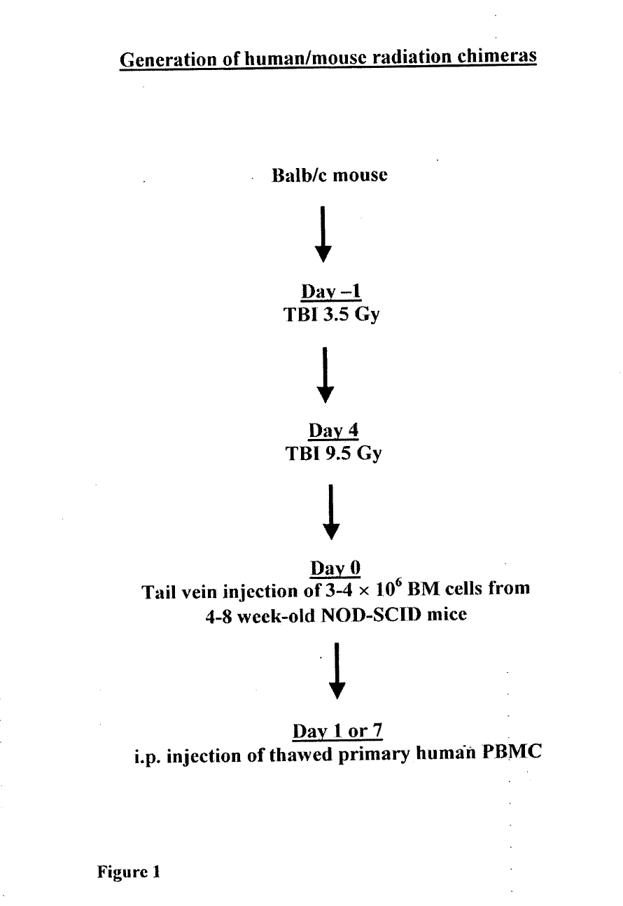

Preparation of human/mouse radiation chimeras: Human/mouse radiation chimeras were prepared as follows (depicted in FIG. 1). Briefly, Balb/c mice were subjected to split-dose TBI via a dose of 3.5 Gy followed by a second dose of 9.5 Gy 3 days later. The day after, the irradiated mice were radioprotected via tail vein injection of 0.2 ml of SCID mouse BM innoculum containing 3-4×10⁶ cells. Bone marrow cells were prepared as previously described [Lubin I, 1995 #71]. Briefly, BM cells were flushed from tibia and femur of 4-8 week old SCID mice, washed and resuspended at 15-20×10⁶ cells/ml in PBS supplemented with streptomycin and penicillin. All mice were obtained from the Weizmann Institute Animal Breeding Center (Rehovot, Israel).

Transplantation of PBLs from B-CLL and T-ALL patients in radioprotected mice: Nine days following radioprotection of mice, as described above, frozen primary PBLs from B-CLL or T-ALL patients were thawed, washed and injected intraperitoneally into the radioprotected mice at various concentrations. As a control, one group of mice was not transplanted with leukemia cells.

For analysis of leukemia cell engraftment, mice were subjected to peritoneal wash with 1% Na-acetate solution daily during Days 1-12 post-leukemia cell injection and recovered cells were analyzed via immunofluorescent flow cytometry, as described below.

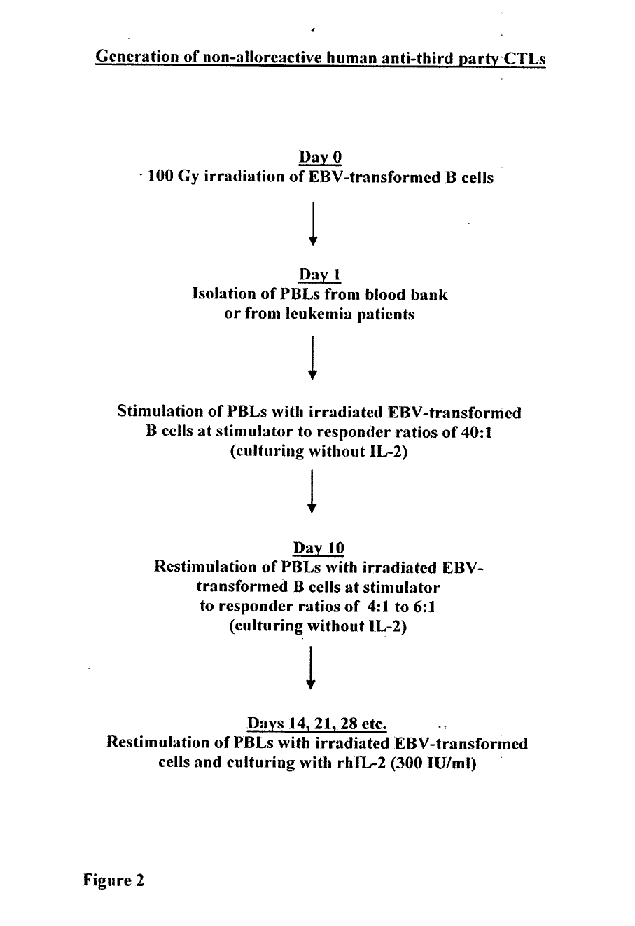

Generation of non-alloreactive human allogeneic anti-third party CTLs: Non-alloreactive human allogeneic anti-third-party CTLs specific for leukemic cells were generated from normal donors by a procedure specifically designed to eradicate anti-host CTL clones, as follows (depicted schematically in FIG. 2).

Briefly, on Day 0, EBV-transformed B cells, were irradiated with a dose of 100 Gy. On Day 1, PBLs were isolated from buffy coats from the blood bank and from advanced-stage B-CLL patients with their written consent (in collaboration with Prof. Alain Berrebi, Kaplan Medical Center, Rehovot). Whole blood or buffy coats were layered onto a cushion of Ficoll-Paque (Pharmacia-Biotech, Sweden), centrifuged at 1800 rpm for 20 minutes and the resulting interlayer containing PBLs was collected, washed twice and resuspended in PBS. The PBLs were then stimulated with the irradiated EBV-transformed B cell line and subsequently restimulated on Day 10 at stimulator to responder ratios of 40:1 and 4:1 to 6:1, respectively. Cells were subjected to IL-2 starvation during the first two weeks of culture, so as to induce apoptosis of potentially anti-host clones being unreactive to the very limited number of EBV-transformed stimulators employed during the first stimulation. Thereafter, cultures were restimulated once a week with an irradiated EBV-transformed cell line and were cultured in medium supplemented with rhIL-2 (300 IU/ml; EuroCentus, Amsterdam, The Netherlands) which was renewed every 2-3 days. This re-stimulation further leads to the selective expansion of EBV antigen-specific having been successfully activated during the IL-2 starvation period.

In vitro killing assays: Standard 4-hour ⁵¹Cr release assays were performed at known effector to target ratios and percent specific cytolysis was measured. Assays were performed in triplicate in round-bottomed tissue culture microtiter plates (Costar) by incubating human allogeneic anti-third party CTLs as effectors with human leukemia cells as ⁵¹Cr-loaded targets. Leukemia cells were stimulated with Con A for 48 hours and then incubated with effectors at 37° C. for 4 hours prior to supernatant harvest for quantitation of ⁵¹Cr release. For control, unstimulated PBMCs from the same donor as that of the effector CTLs were utilized.

In vivo killing assays: One day following radioprotection, mice were engrafted with 100-150×10⁶ PBMCs from B-CLL patients via intraperitoneal injection. One day or one week later, 10-15×10⁶ human allogeneic anti-third party CTLs were administered intraperitoneally into the engrafted mice. After 1 week peritoneal cells were recovered and tested for the presence of B-CLL cells by immunofluorescent flow cytometric analysis, as described below.

Transwell killing assays: Human allogeneic anti-third party CTLs were incubated in 6-well tissue culture plates (Nunclon) with leukemia cells, either together or separated by a membrane preventing contact between cells placed in different compartments but allowing free diffusion of molecules from one compartment to the other (Costar). 5×10⁶ B-CLL cells were incubated with 5×10⁵ human allogeneic anti-third party CTLs and 3×10⁵ T-ALL cells were incubated with 3×10⁶ human allogeneic anti-third party CTLs. Assays were performed in triplicate. B-CLL cells were also incubated as described with autologous CTLs. Following 48 and 72 hours of incubation, cells were recovered from both compartments and analyzed via immunofluorescent flow cytometry, as described below, for presence of B-CLL cells and CTLs.

Immunofluorescent flow cytometric analysis of T-ALL, B-CLL and CTLs: For phenotypic analysis, cells isolated from the peritoneal fluid of engrafted mice or from in vitro cultures were incubated with a mixture of selected monoclonal antibodies labeled with fluorescein isothiocyanate (FITC), peridinin chlorophyll protein (PerCP), phycoerythrin (PE) or cytochrome (Cy) at 4° C. for 20 min. After washing off free antibody, two- or three-color analysis was performed using a FACScan analyzer (Becton-Dickinson). Lymphocytes were gated on the basis of forward- and side-scatter. The following anti-human antibody-fluorescent dye conjugates were used to label surface-expressed molecules: CD3-Cy (Pan T-lymphocyte) was purchased from Pharmingen, CD3-PE (T-cells) and CD45-PerCP (Pan human leukocyte antigen) from Becton Dickinson; CD45-FITC, CD19-FITC, CD20-FITC and CD5-PE from Dako. B-CLL cells, and normal T cells were differentiated by being characterized by CD20/19+CD5+ and CD20/19⁻CD5+ phenotypes, respectively, and T-ALL and normal T cells were differentiated by being characterized by CD34+CD5+ and CD34⁻CD5+ phenotypes, respectively.

Results:

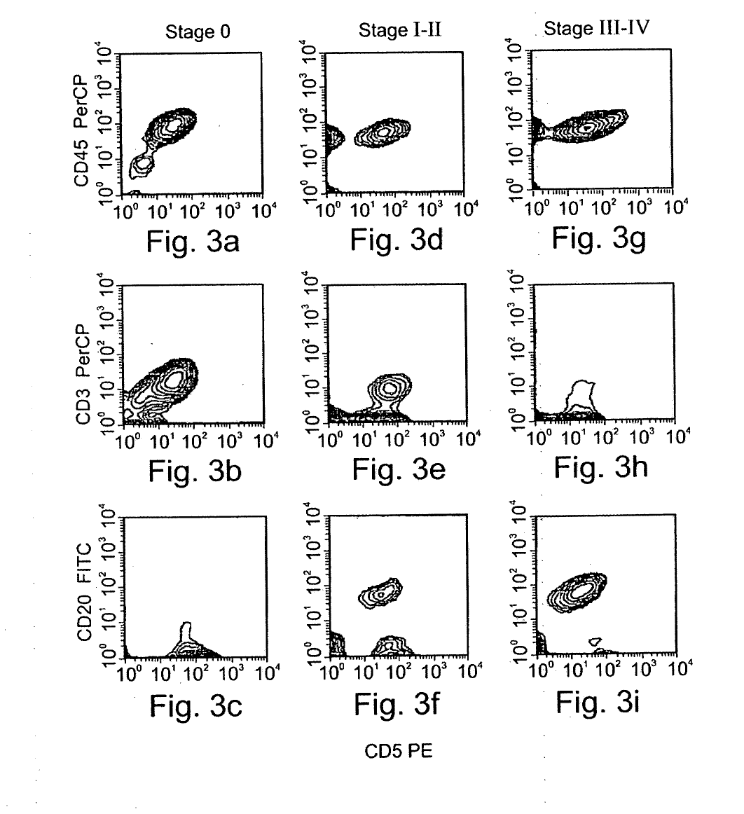

Growth of human leukemic cells in the human/mouse chimera: Engraftment of both acute (T-ALL) and chronic leukemia (B-CLL) cells in lethally irradiated Balb/c mice radioprotected with NOD/SCID BM was analyzed following intra-peritoneal infusion of 100-150×10⁶ cells/mouse. Very high rates of engraftment (80-100% of transplanted mice) of both T-ALL and B-CLL (FIG. 3) cells in the peritoneum were observed.

The phenotypes and frequencies of B-CLL and T-ALL cells present in peritoneal washes was analyzed by immunofluorescent flow cytometry. Four different patterns of engraftment for B-CLL cells were observed, according to the tumor stage of the patient from which the cells were obtained. Transplantation from Stage 0 B-CLL patients led to significant engraftment of human T-cells, representing 19.3±3.4% of the total number of cells recovered from the peritoneum (FIG. 3b), but poor engraftment of leukemia cells, representing only 3.2±1.7% of the total (FIG. 3c). In contrast, in chimeric mice transplanted with cells from patients in Stages III and IV, engraftment levels of leukemic cells were markedly high, with these cells representing 45.8±12.8% of the total number of cells (FIG. 3i) whereas T cells were barely detectable (FIG. 3h). Infusion of B-CLL cells from patients in Stages I-II resulted in engraftment of both T cells (13.2±5.1%; FIG. 3e) and tumor cells (14.2±4.2%; FIG. 3f).

Thus B-CLL cell and autologous T cell engraftment levels were found to positively and negatively, respectively, correlate with disease severity, as previously described (Shimoni et al., 1999, Cancer Res. 1; 59(23):5968-74).

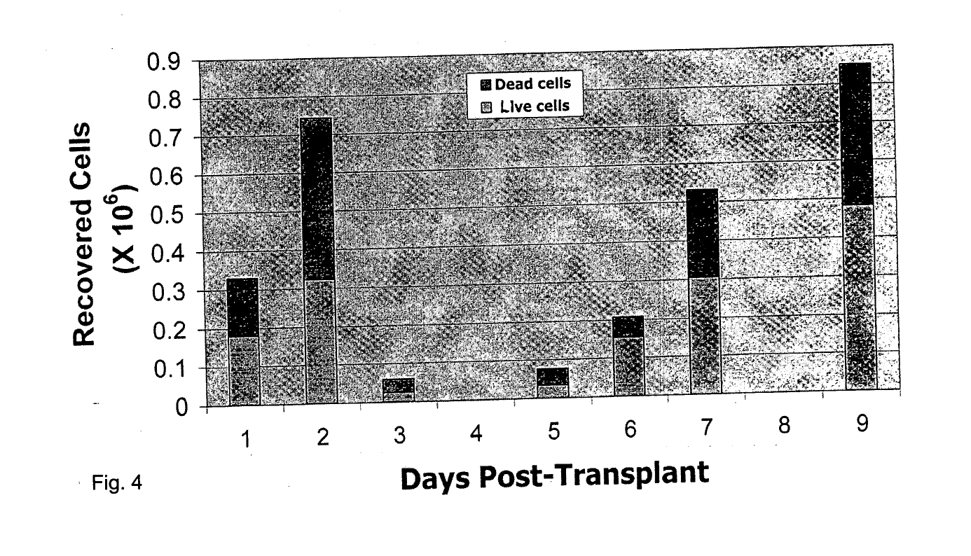

Analysis of T-ALL cell engraftment during the 9-day period post-transplant demonstrated significant levels of engraftment starting on Day 1, a decrease in the number of cells to a nadir on Day 3 followed by an increase to maximal levels on Day 9 (FIG. 4).

Human allogeneic anti-third party CTLs efficiently kill human leukemic cells (“GVL”) in vivo: Following engraftment of leukemia cells in radioprotected mice, killing of leukemia cells by treatment with human allogeneic anti-third party CTLs was analyzed in vitro and in vivo.

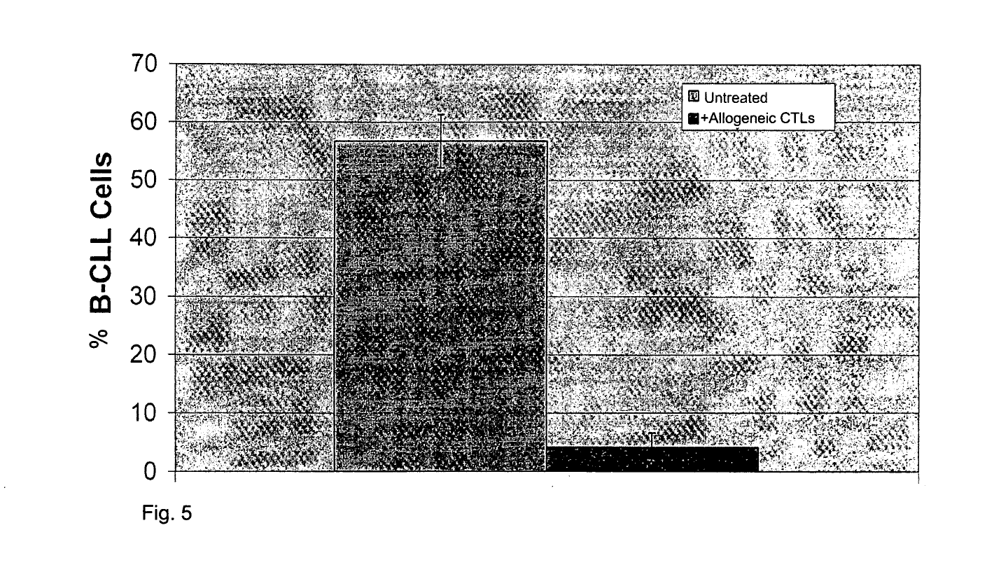

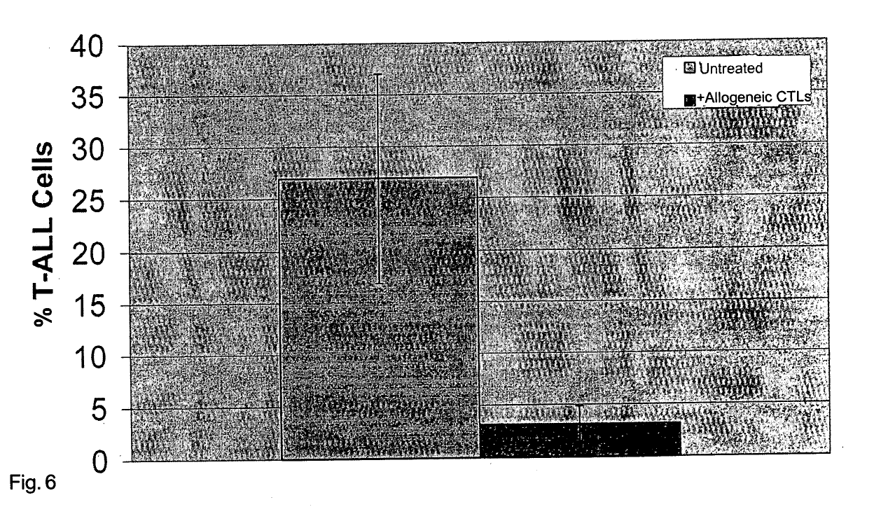

Infusion of human allogeneic anti-third party CTLs in engrafted mice led to marked eradication of human leukemia. As shown in FIGS. 5 and 6, respectively, 93.7% of B-CLL cells and 81.3% of T-ALL cells were eradicated in vivo.

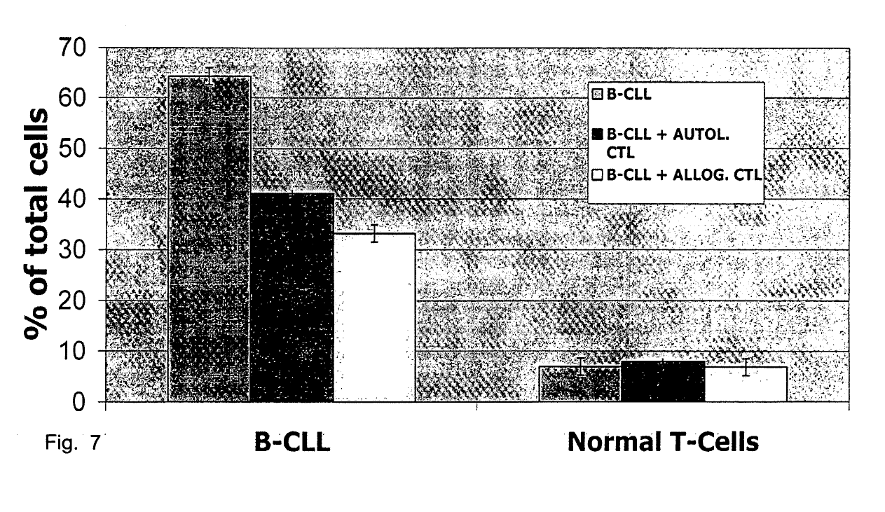

Human allogeneic anti-third party CTLs efficiently kill leukemic cells (“GVL”) but not normal cells (“GVHD”) of leukemia patients in vitro: In vitro co-incubation of human allogeneic anti-third party CTLs with primary B-CLL patient PBLs resulted in significant (>50%) cytolysis of B-CLL cells but not of autologous normal T-cells (FIG. 7). After 48 h of incubation, the 64.3±1.7% fraction of B-CLL cells in cells plated was found to be 33.2±1.7%, whereas the small, ˜7% fraction of B-CLL-autologous normal T-cells in the B-CLL compartment was not altered.

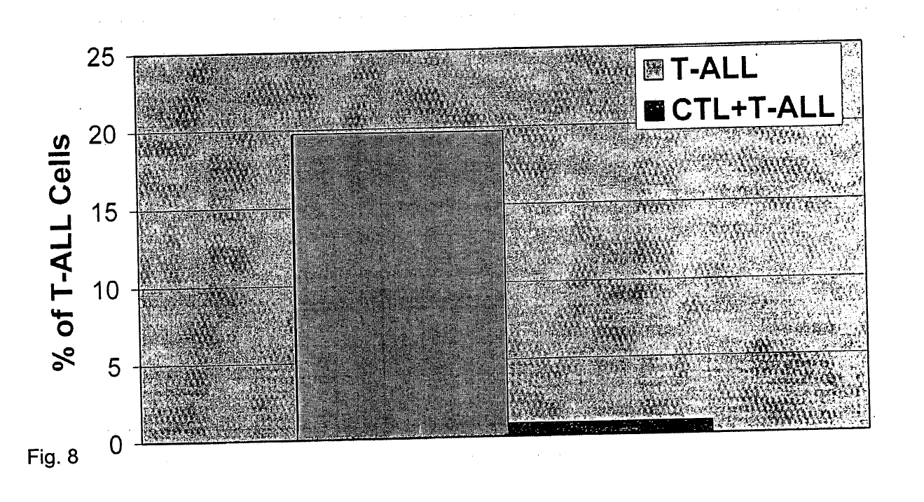

Even more highly efficient in vitro cytolysis of T-ALL cells by human allogeneic anti-third party CTLs was observed. Following 48 h of co-incubation of human allogeneic anti-third party CTLs with primary T-ALL cells the percentage of T-ALL cells was dramatically reduced, from 19.7% to 0.84%, in co-cultures with allogeneic CTLs, representing killing of ˜96% of T-ALL cells (FIG. 8).

B-CLL-autologous anti-third party CTLs efficiently kill B-CLL cells in vitro and in vivo: To verify that the observed killing of B-CLL cells by human allogeneic anti-third party CTLs was not associated with residual alloreactivity, B-CLL-autologous anti-third party CTLs from autologous T cells, generated using the same procedure described above for generating allogeneic anti-third party CTLs, were tested for their ability to kill B-CLL cells.

Results from in vitro studies indicated that after 48 h of incubating B-CLL-autologous anti-third party CTLs with B-CLL cells, the 64.3±1.7% fraction of B-CLL cells in cells plated was found to be 41.1±0.5%, representing a significant reduction (FIG. 7). These levels of cytolysis, being similar to those obtained using allogeneic CTLs, indicate that the observed killing of B-CLL cells by human allogeneic anti-third party CTLs was not associated with residual alloreactivity.

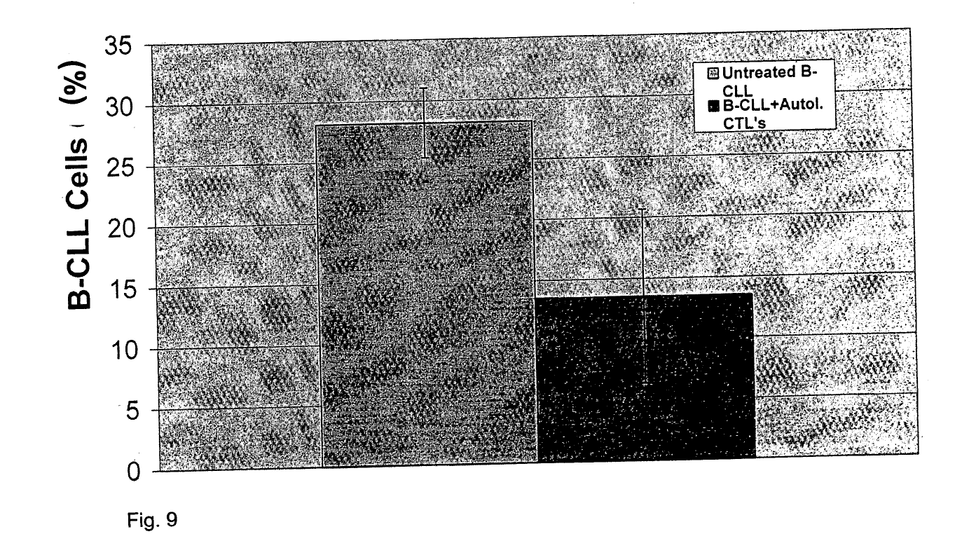

Results from in vivo studies indicated even more efficient killing of B-CLL cells by B-CLL-autologous anti-third party CTLs. Treatment of human/mouse radiation chimeras engrafted with B-CLL cells with B-CLL-autologous anti-third party CTLs resulted in killing of 52% of the B-CLL cells (FIG. 9).

Killing of B-CLL, but not T-ALL cells by anti-third party CTLs requires cell-cell contact between effectors and targets: Experiments to determine whether eradication of leukemic cells by autologous or human allogeneic anti-third party CTLs could be mediated by soluble factors were performed using transwell killing assays.

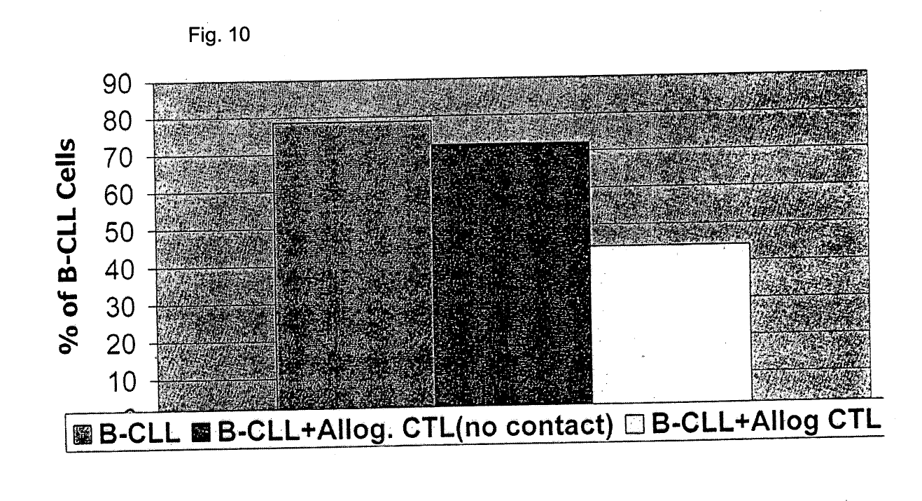

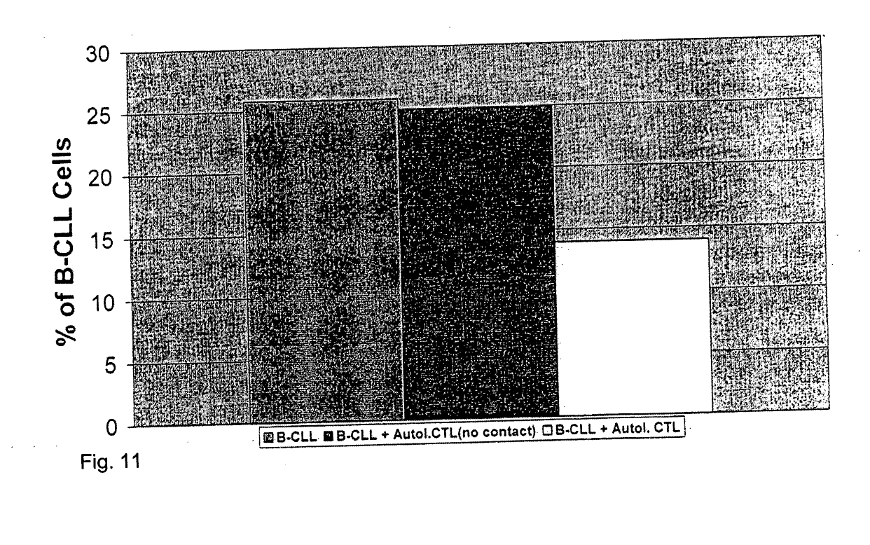

These experiments revealed that contact between human allogeneic or B-CLL-autologous anti-third party CTLs and B-CLL cells was necessary for killing of B-CLL cells. Whereas co-incubation of human allogeneic or B-CLL-autologous anti-third party CTLs and B-CLL cells with cell-cell contact reduced the percentage of B-CLL cells from 78.6% to 44.2%, or from 25.8% to 14.0%, respectively, representing killing of ˜39% or ˜46% of B-CLL cells, respectively, prevention of effector-target cell-cell contact resulted in a negligible reduction of only ˜8% or ˜4% of the B-CLL cells, respectively (FIGS. 10 and 11, respectively).

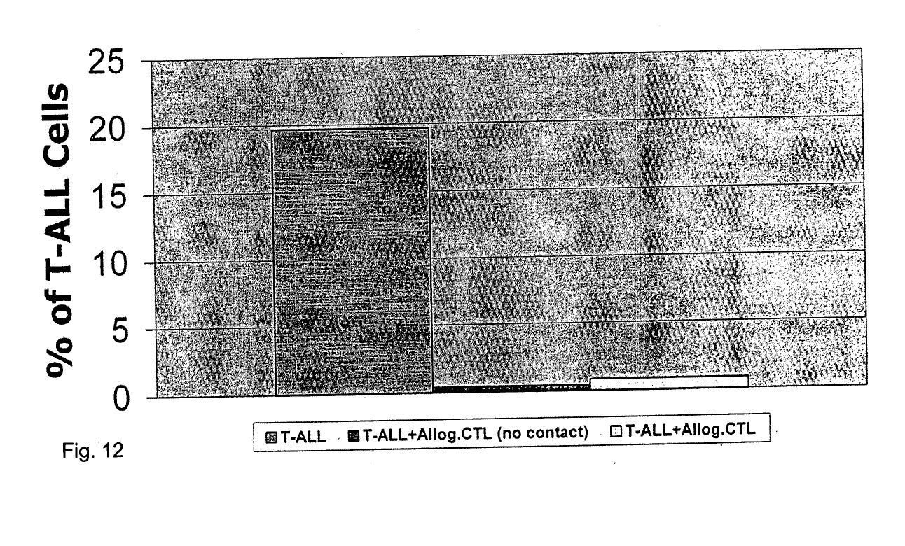

Further experiments showed, however, that human allogeneic anti-third party CTLs were capable of killing T-ALL cells extremely efficiently via soluble factors only. When the T-ALL cells and the human allogeneic anti-third party CTLs were incubated together under conditions either allowing or preventing cell-cell contact with each other, the 19.7% fraction of T-ALL cells was reduced to 0.8% or 0.4%, representing killing of ˜96% or ˜98% of the T-ALL cells, respectively (FIG. 12).

### CONCLUSIONS

These results therefore demonstrate that allogeneic anti-third party CTLs that have been depleted of alloreactivity by IL-2 starvation followed by re-stimulation with allogeneic EBV transformed B lymphocytes, have the capacity to efficiently kill chronic (B-CLL) and acute (T-ALL) leukemia cells both in vivo and in vitro.

These results further demonstrate that T cells isolated from B-CLL patients themselves, can be converted into efficient CTL effectors. Similarly to their allogeneic counterparts, autologous CTLs derived from a leukemia patient, such as a B-CLL patient, are able to eliminate B-CLL cells from the same leukemia patient, both in vivo an in vitro.

Thus, the method of the present invention constitutes a marked improvement over prior art methods of treating malignant and viral diseases using both allogeneic and syngeneic T lymphocytes.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent, patent application was specifically and individually indicated to be incorporated herein by reference. In addition, citation or identification of any reference in this application shall not be construed as an admission that such reference is available as prior art to the present invention.

What is claimed is:

1. A method of treating a disease in a subject by administration of a non-GVHD inducing population of immune effector cells, the method comprising:
(a) co-culturing:
(i) a first cell population comprising cells specifically immunoreactive to an antigen associated with the disease and cells not immunoreactive to said antigen associated with the disease; and
(ii) a second cell population comprising cells being non-syngeneic with the subject and non-syngeneic with said first cell population, said second cell population and a culturing conditions being selected so as to induce proliferation of said cells specifically immunoreactive to an antigen associated with the disease; and
(b) administering immune effector cells resultant from step (a) to the subject, thereby treating the disease without inducing GVHD.

2. The method of claim 1, wherein said co-culturing is effected under conditions suitable for inducing selective proliferation of said cells specifically immunoreactive to said antigen associated with the disease.

3. The method of claim 1, wherein said treating is performed prior to, concomitantly with or following transplantation of allogeneic cells, tissues or organs into the subject.

4. The method of claim 3, wherein said allogeneic cells comprise bone marrow cells.

5. The method of claim 1, wherein the disease is a malignant disease.

6. The method of claim 1, wherein the disease is a viral disease.

7. The method of claim 1, wherein the disease is an autoimmune disease.

8. The method of claim 1, wherein the disease is a leukemia.

9. The method of claim 1, wherein the disease is a myeloid leukemia.

10. The method of claim 1, wherein the disease is a lymphocytic leukemia.

11. The method of claim 1, wherein the disease is an acute leukemia.

12. The method of claim 1, wherein the disease is a chronic leukemia.

13. The method of claim 1, wherein the disease is a T cell leukemia.

14. The method of claim 1, wherein the disease is a B cell leukemia.

15. The method of claim 1, wherein the disease is a T-ALL.

16. The method of claim 1, wherein the disease is a B-CLL.

17. The method of claim 1, wherein the subject is human.

18. The method of claim 1, wherein said administration is effected via intraperitoneal injection.

19. The method of claim 1, wherein said administration is effected via intravenous injection.

20. The method of claim 1, wherein said first cell population is derived from a donor being allogeneic with the subject.

21. The method of claim 1, wherein said first cell population is derived from a donor being syngeneic with the subject.

22. The method of claim 1, wherein said first cell population is derived from the subject.

23. The method of claim 1, wherein said first cell population comprises PBMCs.

24. The method of claim 1, wherein said first cell population comprises PBLs.

25. The method of claim 1, wherein said first cell population comprises cells derived from a lymphoid organ.

26. The method of claim 25, wherein said lymphoid organ is selected from the group consisting of bone marrow, spleen, lymph node, Peyer's patch and thymus.

27. The method of claim 1, wherein said first cell population comprises a population of cells differentiated in vitro.

28. The method of claim 1, wherein said first cell population comprises a population of genetically transformed cells.

29. The method of claim 1, wherein said cells specifically immunoreactive to an antigen associated with the disease comprise T lymphocytes.

30. The method of claim 29, wherein said T lymphocytes comprise helper T lymphocytes.

31. The method of claim 29, wherein said T lymphocytes comprise CTLs.

32. The method of claim 1, wherein said cells not immunoreactive to said antigen associated with the disease comprise T lymphocytes.

33. The method of claim 32, wherein said T lymphocytes comprise helper T lymphocytes.

34. The method of claim 32, wherein said T lymphocytes comprise CTLs.

35. The method of claim 1, wherein said cells specifically immunoreactive to an antigen associated with the disease are specifically immunoreactive to an antigen associated with the disease as a result of genetic transformation.

36. The method of claim 1, wherein said cells specifically immunoreactive to an antigen associated with the disease comprise genetically transformed natural killer cells.

37. The method of claim 1, wherein said cells not immunoreactive to said antigen associated with the disease are T lymphocytes.

38. The method of claim 37, wherein said T lymphocytes comprise helper T lymphocytes.

39. The method of claim 37, wherein said T lymphocytes comprise CTLs.

40. The method of claim 1, wherein said second cell population comprises cells being infected with a virus.

41. The method of claim 40, wherein said virus is Epstein-Barr virus.

42. The method of claim 1, wherein said second cell population comprises antigen presenting cells.

43. The method of claim 1, wherein said second cell population comprises B cells.

44. The method of claim 1, wherein said co-culturing is effected under conditions of IL-2 starvation.

45. The method of claim 2, wherein said conditions suitable for selective proliferation of said cells specifically immunoreactive to said antigen associated with the disease comprise IL-2 supplementation.

46. The method of claim 1, wherein said cells specifically immunoreactive to said antigen associated with the disease are T-lymphocytes and further wherein said second cell population are EBV infected B-lymphocytes.

47. A method of treating a disease in a subject by administration of a non-GVHD inducing population of T lymphocytes, the method comprising:
(a) co-culturing:
(i) a first cell population comprising T-lymphocytes specifically immunoreactive to an antigen associated with the disease and T-lymphocytes not immunoreactive to said antigen associated with the disease; and
(ii) a second cell population comprising EBV-infected B-lymphocytes being non-syngeneic with the subject and non-syngeneic with said first cell population;
said co-culturing being effected under conditions selective for killing cells of said first cell population not immunoreactive to said antigen associated with the disease, while being further selective for inducing proliferation of said T-lymphocytes specifically immunoreactive to said antigen associated with the disease; and
(b) administering viable T lymphocytes resultant from step (a) to the subject, thereby treating the disease without inducing GVHD.

48. The method of claim 47, wherein said second cell population comprises cells expressing or displaying said antigen associated with the disease.

49. The method of claim 47, wherein the disease is leukemia.
